(19) United States
(12) Patent Application Publication
(10) Pub. No.: US 2022/0296534 A1
(43) Pub. Date: Sep. 22, 2022

(54) SCYLLO-INOSITOL AND B-CELL MEDIATED DISORDERS

(71) Applicant: SOCIETE DES PRODUITS NESTLE S.A., Vevey (CH)

(72) Inventors: JEAN-PHILIPPE GODIN, Ithaca, NY (US); IRMA SILVA ZOLEZZI, Linwood, NJ (US); JOSE MANUEL RAMOS NIEVES, Lausanne (CH); MARINE KRAUS, Vuarrens (CH)

(21) Appl. No.: 17/753,316

(22) PCT Filed: Aug. 28, 2020
(86) PCT No.: PCT/EP2020/074070

§ 371 Date: Feb. 28, 2022

(30) Foreign Application Priority Data
Aug. 30, 2019 (EP) 19194599.7

(51) Int. Cl. A61K 31/047; A61K 35/60; A61K 35/745; A61K 35/747; A61P 3/10

(52) U.S. Cl. CPC A61K 31/047; A61K 35/60; A61K 35/745; A61K 35/747; A61P 3/10

(57) ABSTRACT

The present invention relates generally to the field of β-cell mediated disorders. In particular, the present invention relates to scyllo-inositol for use in the treatment or prevention of disorders linked to an impaired function of insulin secreting β-cells. The present invention further relates to scyllo-inositol for use in the treatment or prevention of a condition or symptom associated with such disorders in a subject.

The present invention relates generally to the field of β-cell mediated disorders. In particular, the present invention relates to scyllo-inositol for use in the treatment or prevention of disorders linked to an impaired function of insulin secreting β-cells. The present invention further relates to scyllo-inositol for use in the treatment or prevention of a condition or symptom associated with such disorders in a subject.

Type 2 diabetes mellitus (T2D) is a significant disease in humans and a major health issue worldwide. Prevalence of T2D has increased dramatically in the last decades with 1 million people reported to be diagnosed with T2D in 1994, increasing to 382 million in 2013 and with prediction of 595 million by 2035. T2D is the main cause of death for approximately 1.5 million people annually and is a risk factor for cardiovascular disease that is the leading cause of 13 million deaths worldwide every year, accounting for 25% of all deaths. This disease is not only prevalent in Western countries, North American and Oceania, but its prevalence is growing in Asian countries, in particular China as well.

T2D is a progressive metabolic disorder characterized by insulin resistance and hyperglycemia, the result of a lack or cessation of morphological and functional β-cell compensation in response to an elevated insulin demand during insulin resistance. Indeed, plasma insulin levels are predominantly the product of the morphological mass of insulin producing β-cells in the pancreatic islets of Langerhans and the functional status of each of these β-cells. In many individuals, genetic predisposition and/or an unhealthy lifestyle leads to an increased insulin resistance, which is typically met by massive functional and moderate morphological compensation to maintain normoglycemia, thus increasing the workload of each β-cell. This typically successful compensation is mainly driven by functional alterations, which outperform mass adaptation in rate and extent.

In prediabetic people, chronic glucose intolerance and elevated blood glucose levels continuously exacerbate β-cell workload and stress, culminating in cellular exhaustion, cell death, and clinical manifestation of hyperglycemia. Thereafter, uncontrolled hyperglycemia, often in concert with other cytotoxic factors, leads to accelerated β-cell mass loss and functional deterioration in overt diabetic patients. Therefore, the progressive β-cell failure (in mass and function) during the prediabetes phase is the major factor responsible for the occurrence of T2D. Nonetheless, the precise contribution of β-cell mass and function to the pathogenesis of diabetes as well as the underlying mechanisms are still unclear. To give an example, β-cell function is already decreased by 50% in subjects with fasting glucose or 2 h plasma glucose levels at the upper limit of the normal range (respectively 95-100 mg/dL, and 130-139 mg/dL) whereas subjects diagnosed as T2D have already lost over 80% of their β-cell function.

Currently, most clinical treatments of T2D either target insulin resistance or aim to elevate insulin levels by increasing β-cell function. In light of a potential exhaustive effect on β-cells, more basic research turns to study mechanisms to regenerate β-cell mass or to preserve β-cell function. Indeed, in T2D, the protection and recovery of β-cell function should be a main treatment and prevention target. Therefore, drugs/ingredients able to stimulate or enhance insulin secretion will moderate hyperglycemia and then reduce the occurrence of later complication of the disease.

Other prior art methods for treating glucose tolerance include myo-inositol and d-chiro-inositol. Inositol is a group of compounds containing six-hydroxyl groups in a cyclohexane. The epimerization of these six-hydroxyl groups produces nine stereoisomers named scyllo-inositol, cis-inositol, epi-inositol, neo-inositol, D-chiro-inositol, L-chiro-inositol, muco-inositol, allo-inositol and myo-inositol. Myo-inositol is the most common one in nature. It plays an important role in various cellular processes, in membrane as phospholipids and as phosphatidylinositol and as secondary messengers via inositol phosphates and possibly inositol glycans.

Myo-inositol supplementation lowers postprandial glucose, to improve insulin sensitivity in women with polyscytic ovary syndrome [Gynecol Endocrinol, 2012. 28(6): p. 440-2; Int J Endocrinol, 2016. 2016: p. 1849162; Trends Endocrinol Metab, 2018. 29(11): p. 768-780]. In several randomized control trials myo-inositol was also assessed in pregnant women at risk of gestational diabetes mellitus [Diabetes Care, 2013. 36(4): p. 854-857; J Matern Fetal Neonatal Med, 2013. 26(10): p. 967-72; Diabet Med, 2011. 28(8): p. 972-5], and in patients with metabolic syndrome and type 2 diabetes [Menopause, 2011. 18(1): p. 102-4; Int J Endocrinol, 2016. 2016: p. 9132052] with some beneficial effects.

Dietary and lifestyle changes, including healthier dietary habits and increased exercise, can be very efficient in preventing or treating prediabetes, type II diabetes and/or gestational diabetes mellitus, however, patient compliance is often an issue. Drugs such as biguanides and thiazolidinediones may also be used. However, many of these have undesirable side effects. Moreover, there is no practice of giving such drugs to prevent prediabetes and many are unsuitable for use during pregnancy. Accordingly, there is a need to find alternative ways to treat or prevent prediabetes, type II diabetes, gestational diabetes mellitus and/or a condition associated with any of the foregoing in a subject. In particular, there is a need to find ways that may not suffer from one or more of the drawbacks of the prior art.

Any reference to prior art documents in this specification is not to be considered an admission that such prior art is widely known or forms part of the common general knowledge in the field.

Accordingly, there is a need to find alternative ways to treat or prevent disorders linked to an impaired function of insulin secreting β-cells and/or a condition or symptom associated with such disorders in a subject. Such disorders may be for example prediabetes, type II diabetes, gestational diabetes mellitus as far as they are linked to an impaired function of insulin secreting β-cells. In particular there is a need to find ways that may not suffer from one or more of the drawbacks of the prior art.

It would therefore be desirable to provide a compound and/or a composition for use in the treatment or prevention of disorders linked to an impaired function of insulin secreting β-cells and/or a condition or symptom associated with such disorders in a subject. Preferably, such a compound and/or a composition for such a use is without one or more of the disadvantages listed above; or at least to provide a useful alternative.

The object of the present invention was it, hence, to enrich or improve the state of the art and in particular, to provide a compound and/or a composition for use in the treatment or prevention of disorders linked to an impaired function of insulin secreting β-cells and/or a condition or symptom associated with such disorders in a subject.

The inventors were surprised to see that the object of the present invention could be achieved by the subject matter of the independent claim. The dependent claims further develop the idea of the present invention.

Accordingly, the present invention provides scyllo-inositol for use in the treatment or prevention of disorders linked to an impaired function of insulin secreting β-cells and/or conditions or symptoms associated with such disorders in a subject.

As used in this specification, the words “comprises”, “comprising”, and similar words, are not to be interpreted in an exclusive or exhaustive sense. In other words, they are intended to mean “including, but not limited to”.

So far, therapeutic effects of scyllo-inositol were reported in the context of neurological disorders. Indeed, scyllo-inositol concentrations in the cerebrospinal fluid (CSF) and brain seemed to increase after its administration, and preclinical data supported that a sustained elevated level of scyllo-inositol in the brain seemed necessary for the therapeutic efficacy. Thus, scyllo-inositol has been clinically studied for the treatment of Alzheimers' disease (AD). Scyllo-inositol alone has been shown to prevent the formation of insoluble amyloid fibers that are increased during Alzheimer's disease [Adv Pharmacol, 2012. 64: p. 177-212; Neurology, 2011. 77(13): p. 1253-62; Expert Opin Pharmacother, 2017. 18(6): p. 611-620], and to inhibit the neuronal aggregation of alpha-synuclein (protein) in Parkinson's disease.

Overall, in humans, scyllo-inositol is completely and rapidly absorbed (the mean time to reach the maximum level after ingestion is about 3.8 h) after ingestion. In vivo steady state (in healthy men) of scyllo-inositol is obtained after 5-6 days of continuous ingestion (2 g/12 h during 10 days). A series of scyllo-inositol derivatives have been synthesized which contain deoxy, fluoro, chloro, methoxy and guanidine substitutions. Guanidine-scyllo-inositol derivative was tested in transgenic AD mice (dose given 25-30 mg/day/mouse or 10 μM for cells experiments).

Scyllo-inositol level in plasma and urine fluids was rarely described in peer-reviewed papers but the highest level of scyllo-inositol in the body is in the brain. As example, in gray matter, average scyllo-inositol level was about 0.78 mM. Presently, it is believed that scyllo-inositol enters into cells by using SMIT1/2 transporters. However, no specific studies looked at scyllo-inositol efflux from cells. This gap may relate to the poor knowledge regarding the endogenous function and degradation of scyllo-inositol.

The present inventors were surprised to find that scyllo-inositol has a unique and potent effect to stimulate insulin secretion, one key function of β-cells. Knowing that β-cell dysfunction has been repeatedly observed in people with prediabetes conditions [Curr Diabetes Rev, 2016. 12(1): p. 30-41; Diabetologia, 2008. 51(5): p. 853-61; Diabetes Care, 2009. 32(3): p. 439-44], and is the hallmark of T2D and type-1 diabetes (T1D) conditions, the subject matter of the present invention will be effective to treat or prevent disorders linked to an impaired function of insulin secreting β-cells and/or a condition or symptom associated with such disorders, for example during prediabetes, T2D and T1D conditions.

The present invention relates to the treatment and prevention of disorders linked to an impaired function of insulin secreting β-cells and/or a condition or symptom associated with such disorders. In a further aspect, the invention relates to scyllo-inositol for use in the treatment or prevention of disorders linked to an impaired function of insulin secreting β-cells and/or a condition or symptom associated with such disorders in a subject.

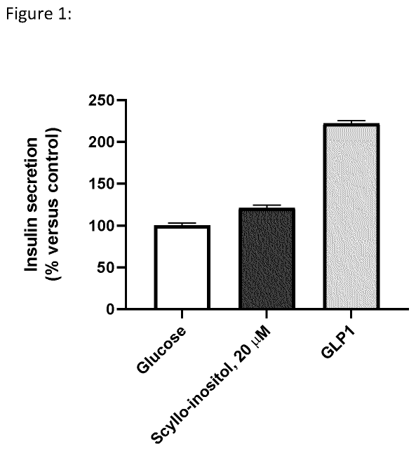

FIG. 1 shows the glucose stimulated insulin secretion of scyllo and myo-inositols at 20 μM w with glucose and GLP1 as controls in INS1-cells.

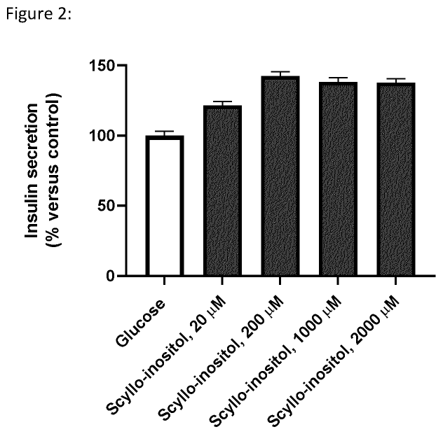

FIG. 2 shows the glucose stimulated insulin secretion of scyllo-inositol at different concentrations (from 20 to 2000 μM) in INS1-cells.

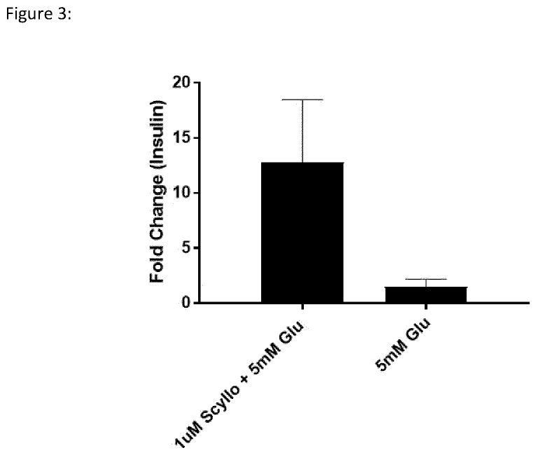

FIG. 3 shows the glucose stimulated insulin secretion in human islets with scyllo-inositol at 1 μM on 5 mM glucose stimulation.

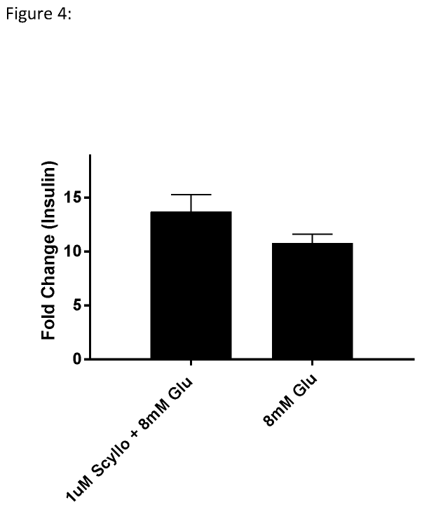

FIG. 4 shows the glucose stimulated insulin secretion in human islets with scyllo-inositol at 1 μM on 8 mM glucose stimulation.

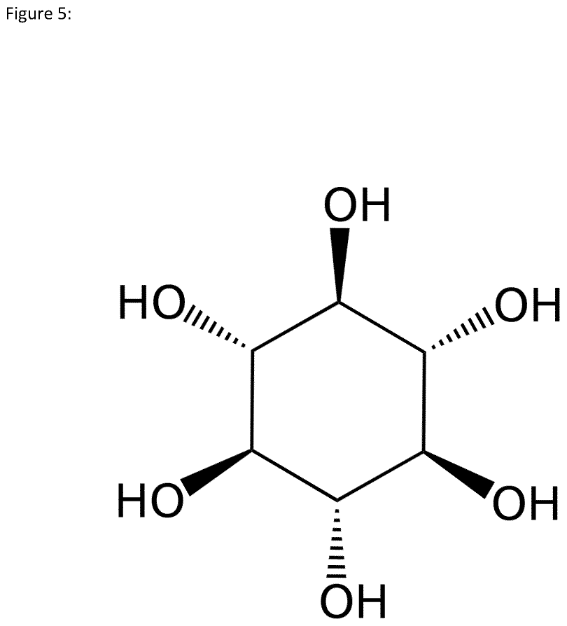

FIG. 5 shows the structure of scyllo-inositol.

Consequently, the present invention relates in part to scyllo-inositol. The present invention also relates to scyllo-inositol for use as a medicament. The present invention further relates to scyllo-inositol for use in the treatment or prevention of disorders linked to an impaired function of insulin secreting β-cells and/or a condition or symptom associated with such disorders. The present invention also relates to the use of scyllo-inositol for the manufacture of a composition for the treatment or prevention of disorders linked to an impaired function of insulin secreting β-cells and/or a condition or symptom associated with such disorders.

In one embodiment, the subject matter of the present invention relates to scyllo-inositol for use in the treatment or prevention of disorders linked to an impaired function of insulin secreting β-cells and/or a condition or symptom associated with such disorders in a subject.

The term “treat” or “treatment” as used herein encompasses amelioration and/or alleviation of a disorder i.e. the amelioration and/or alleviation of the symptoms of a disorder or conditions associated with such disorders. It may for example encompass the reduction of the severity of a disorder in a subject.

The term “prevent” or “prevention” as used herein refers to the prevention of the occurrence, or reduction of the risk of the occurrence, of a disorder or conditions associated with such disorders in a subject.

The term “subject” as used herein refers to a mammal and more particularly a cat, a dog or a human. The human may be an adult, child or infant. The human may be a woman, for example, a woman who is trying to get pregnant, who is pregnant, or who is lactating. The subject may also be the offspring of a woman who is trying to get pregnant, who is pregnant, or who is lactating.

In an embodiment of the invention the subject is a mammal selected from the group consisting of a cat, a dog and, a human.

The term “scyllo-inositol” as used herein refers to (1r,2r,3r,4r,5r,6r)-Cyclohexane-1,2,3,4,5,6-hexol.

The present inventors propose scyllo-inositol for use in the treatment or prevention of disorders linked to an impaired function of insulin secreting β-cells.

T2D is a progressive metabolic disorder characterized by insulin resistance and hyperglycemia, the result of a lack or cessation of morphological and functional beta cell compensation in response to an elevated insulin demand during insulin resistance. Plasma insulin levels are predominantly the product of the morphological mass of insulin producing beta cells in the pancreatic islets of Langerhans and the functional status of each of these beta cells. In many individuals, genetic predisposition and unhealthy lifestyle lead to an increased insulin resistance, which is typically met by massive functional and moderate morphological compensation to maintain normoglycemia, thus increasing the workload of each beta cell. This typically successful compensation is mainly driven by functional alterations, which outperform mass adaptation in rate and extent. In this prediabetic phase, chronic glucose intolerance and elevated blood glucose levels continuously exacerbate beta cell workload and stress, culminating in cellular exhaustion, cell death, and clinical manifestation of hyperglycemia. Thereafter, uncontrolled hyperglycemia, often in concert with other cytotoxic factors, leads to accelerated beta cell mass loss and functional deterioration in overt diabetic patients. Nonetheless, the precise contribution of beta cell mass and function to the pathogenesis of diabetes as well as the underlying mechanisms are still unclear. Currently, most clinical treatments of T2D either target insulin resistance or aim to elevate insulin levels by increasing beta cell function. In light of a potential exhaustive effect on beta cells, more basic research turns to study mechanisms to regenerate beta cell mass or to preserve beta cell function. Indeed, in T2D, the protection and recovery of beta cell function should be a main treatment and prevention target. Therefore, drugs/ingredients able to stimulate or enhance insulin secretion will moderate hyperglycemia and then reduce the occurrence or later complication of the disease.

Indeed, recent studies have shown that in type 1 diabetes (T1D) and T2D impairment of β-cell function is an early feature of the disease pathogenesis. This is in contrast to the later clinical manifestations of these disorders where the pathogenesis is linked to an actual decrease in β-cell mass [Mol Metab. 2017 September; 6(9): 943-957].

Consequently, rather than addressing β-cell mass replacement as in late stage therapies, the protection and recovery of β-cell function as proposed by the present inventors will be effective in the treatment or prevention of disorders linked to an impaired function of insulin secreting β-cells, such as early stage T2D.

In accordance with the present invention, the scyllo-inositol may be for use in the treatment or prevention of disorders linked to an impaired function of insulin secreting β-cells and/or a condition or symptom associated with such disorders in a subject by protecting and/or recovering of β-cell function.

In one embodiment of the present invention the disorder linked to an impaired β-cell function is selected from the group consisting of type 1 diabetes mellitus, type 2 diabetes mellitus, prediabetes, gestational diabetes mellitus, and polycystic ovary syndrome.

As in accordance with the present invention scyllo-inositol can be used to specifically address an impaired β-cell function, one embodiment of the present invention relates to the prevention of disorders linked to an impaired β-cell function, for example, selected from the group consisting of type 2 diabetes mellitus, prediabetes, gestational diabetes mellitus, and polycystic ovary syndrome.

In one embodiment of the present invention, the disorder linked to an impaired β-cell function is early stage type 2 diabetes mellitus. In another embodiment of the present invention, the disorder linked to an impaired β-cell function is prediabetes.

Hence, the subject matter of the present invention is particular useful for treating or preventing early features of the diabetes disease pathogenesis. Hence, in accordance with the present invention the scyllo-inositol may be for use in the treatment or prevention of disorders linked to an impaired function of insulin secreting β-cells in a subject, wherein the disorders linked to an impaired function of insulin secreting β-cells is prediabetes, gestational diabetes and/or early stage type 2 diabetes mellitus.

The scyllo-inositol may be used in accordance with the present invention for any subject suffering from or at risk of developing disorders linked to an impaired function of insulin secreting β-cells and/or a condition or symptom associated with such disorders. Increasingly, not only humans suffer from such disorders or are at risk of developing them, but other mammals, in particular pets, do as well. Hence, in one embodiment of the present invention, the subject may be a mammal. For example, the mammal may be selected from the group consisting of a cat, a dog and a human.

Gestational diabetes mellitus is a disorder that affects pregnant woman.

Hence, in one embodiment of the present invention the subject is a female who is trying to get pregnant, is pregnant or who is lactating for her offspring.

Accordingly, in accordance with the present invention the scyllo-inositol may be for use in the treatment or prevention of disorders linked to an impaired function of insulin secreting β-cells in a subject, wherein the disorders linked to an impaired function of insulin secreting β-cells is prediabetes, gestational diabetes and/or early stage type 2 diabetes mellitus and wherein the subject is a female who is trying to get pregnant, is pregnant or who is lactating for her offspring.

For example, the scyllo-inositol may be for use in the treatment or prevention of disorders linked to an impaired function of insulin secreting β-cells in a subject, wherein the disorders linked to an impaired function of insulin secreting β-cells is pre-gestational diabetes and/or gestational diabetes and wherein the subject is a female who is trying to get pregnant, is pregnant or who is lactating for her offspring.

In an embodiment of the present invention the disorder is gestational diabetes mellitus or a condition associated therewith and the subject is a woman who is trying to get pregnant, is pregnant or who is lactating or is her offspring.

If the scyllo-inositol is administered to a human subject desiring to get pregnant, it may be administered during at least 1, 2, 3 or 4 months preceding the pregnancy or desired pregnancy. If it is administered to a pregnant subject, it may be administered throughout or partially throughout the pregnancy e.g. for at least 4, at least 8, at least 12, at least 16, at least 20, at least 24, at least 28, or at least 36 weeks depending on the gestational period of the subject. Administration may also continue throughout or partially throughout the lactation period of said subject.

Since the risk of gestational diabetes mellitus increases in the second and third trimester of pregnancy, administration may be particularly beneficial in the second and third trimester of pregnancy for the prevention or treatment of GDM, or the prevention of a condition associated therewith in a pregnant subject or its offspring.

In an embodiment of the invention the scyllo-inositol is administered in at least the second and/or third trimester of pregnancy wherein the subject is a pregnant woman or her offspring.

In the framework of the present invention, the scyllo-inositol may be administered to a subject in any effective amount. An effective amount may be any amount that improves, by any degree an impaired function of insulin secreting β-cells. It is well within the purview of the skilled person to determine an effective amount. An effective amount may, for example, be determined by testing the effect of an amount on a subject's fasting glucose plasma concentration, or fasting HbA1c concentration, or on glycemic levels at 1 hr, 2 hr during an OGTT. An effective amount should improve a subject's fasting glucose plasma concentration and/or HbA1c concentration, and/or lower a subject's glycemic response, in particular, if the subject is suffering from prediabetes, type II diabetes or gestational diabetes mellitus.

Scyllo-inositol may for example to be administered to a subject in an amount of up to 4000 mg per day, up to 2000 mg per day, up to 1000 mg per day, up to 500 mg per day, up to 250 mg per day, up to 150 mg per day, up to 125 mg per day, up to 100 mg per day, up to 80 mg per day, up to 70 mg per day, up 50 mg per day, up to 20 mg per day, up to 10 mg per day or up to 1 mg per day.

In an embodiment of the invention the scyllo-inositol is administered to a subject in an amount up to 1 g per day. In a further embodiment of the present invention the scyllo-inositol is to be administered in an amount of 0.1 to 500 mg per day. The inventors currently believe that in certain circumstances it may be advisable that the scyllo-inositol is to be administered in an amount in the range of 10-100 mg scyllo-inositol per day, for example in the range of 40-60 mg scyllo-inositol per day.

Typically, an effective amount will depend on the type, age, size, health status, lifestyle and/or genetic heritage of the subject. The effective amount may be split into several smaller amounts and administered throughout the day so as the total daily intake is the effective amount.

Consequently, in one embodiment of the present invention, the scyllo-inositol may be to be administered to a subject in an amount of up to 0.001 g per kg body weight per day. Alternatively, the scyllo-inositol may be to be administered to a subject in an amount of up to 0.01 g per kg body weight per day, up to 0.008 g per kg body weight per day, up to 0.006 g per kg body weight per day, up to 0.004 g per kg body weight per day, up to 0.002 g per kg body weight per day, or up to 0.0005 g per kg body weight per day.

Scyllo-inositol may be used in accordance with the present invention as scyllo-inositol or in its phosphate form, e.g. scyllo-inositol bis, tris or hexakisphosphate; in meso or racemic forms. Other derivatives of scyllo-inositol could also be employed, for example, fluorinates, C-methyl and, deoxy-scyllo-inositols. Usually, scyllo-inositol will be used in accordance with the present invention.

Scyllo-inositol is commercially available, for example, from Sigma-Aldrich or from other ingredient suppliers.

Scyllo-inositol may also be produced using a variety of methods. Such methods for the manufacturing of scyllo-inositol are disclosed in the art [Angew. Chem. Int. Ed. 55, 16-14-1650; which is hereby incorporated by reference in its entirety]. Alternatively, scyllo-inositol may be produced from myo-inositol in a bio-conversion process using microorganisms such as Pseudomos and Acetobacter. For example, scyllo-inosose may be produced from myo-inositol in a bio-conversion process using micro-organisms belonging to the genus Acetobacter and, the produced Scyllo-inosose may subsequently be enzymatically reduced to scyllo-inositol.

Several symptoms are associated with an impaired β-cell function. These symptoms are well known to the person skilled in the art. The subject matter of the present invention may be used to treat or prevent such symptoms associated with an impaired β-cell function. In one embodiment of the present invention, the symptom associated with an impaired β-cell function may be selected from the group consisting of un-usual hunger, increased thirst, un-usual bed-wetting, un-usual mood changes, irritability, fatigue, frequent urination, blurred eye sight, un-intended weight-loss, overweightness, obesity, or combinations thereof.

Numerous conditions are associated with disorders linked to an impaired β-cell function. Such conditions are well known to the person skilled in the art. The subject matter of the present invention may be used to treat or prevent such conditions associated with disorders linked to an impaired β-cell function. In one embodiment of the present invention the condition associated with disorders linked to an impaired β-cell function may be selected from the group consisting of nephropathy, heart disease, neuropathy, blood vessel disease, skin infections, complications during pregnancy, impaired vision due to damages in the blood vessels of the retina, foot complications, cardiovascular diseases, fatty liver diseases, or combinations thereof.

Numerous conditions are associated with prediabetes, type II diabetes and/or gestational diabetes linked to an impaired β-cell function. In an embodiment of the invention the condition associated with gestational diabetes mellitus linked to an impaired β-cell function is selected from the group consisting of preterm and caesarean delivery, birth injury to the mother or baby, shoulder dystocia, macrosomia, excessive offspring blood glucose concentration, excess weight/adiposity and associated metabolic disorders e.g. type II diabetes, fatty liver disease and obesity immediately after birth and later in the life of the offspring, and an increased risk for the mother of having or developing type 2 diabetes immediately after birth and/or later in life.

Scyllo-inositol may be effective at preventing and/or treating prediabetes, type II diabetes and gestational diabetes mellitus linked to an impaired β-cell function in subject's who are at risk of suffering from one or more of these conditions. Accordingly, in an embodiment of the invention the subject e.g. the human subject, is at risk of suffering from pre-diabetes, type II diabetes or gestational diabetes mellitus. This may be the case, for example, because they do not have a favorable metabolic profile and/or have a history or family history of one or more of these disorders.

Gestational diabetes mellitus is a disorder that affects pregnant woman. Accordingly, in another embodiment of the present invention the disorder linked to an impaired function of insulin secreting β-cells is gestational diabetes mellitus or a condition associated therewith and the subject is a woman who is trying to get pregnant, is pregnant or who is lactating or is her offspring.

If the scyllo-inositol is to be administered to a human subject desiring to get pregnant, it may be to be administered during at least 1, 2, 3 or 4 months preceding the pregnancy or desired pregnancy. If the scyllo-inositol is to be administered to a pregnant subject, it may be administered throughout or partially throughout the pregnancy e.g. for at least 4, at least 8, at least 12, at least 16, at least 20, at least 24, at least 28, or at least 36 weeks depending on the gestational period of the subject. Administration may also continue throughout or partially throughout the lactation period of said subject.

Since the risk of gestational diabetes mellitus increases in the second and third trimester of pregnancy, administration may be particularly beneficial in the second and third trimester of pregnancy for the prevention or treatment of gestational diabetes mellitus linked to an impaired function of insulin secreting β-cells, or the prevention of a condition associated therewith in a pregnant subject or its offspring.

In an embodiment of the invention, the scyllo-inositol is to be administered in at least the second and/or third trimester of pregnancy wherein the subject is a pregnant woman or her offspring.

Probiotics have been found to improve the gut barrier function and to help nutrients pass through the gut. Administrating probiotics in combination scyllo-inositol may therefore enhance the absorption of this compound. Accordingly, in an embodiment the scyllo-inositol is administered in combination with a probiotic, for example a probiotic selected from the group consisting of Lactobacillus rhamnosus GG (CGMCC 1.3724), Bifidobacterium lactus BB-12 (CNCM I-3446), or a combination thereof.

Lactobacillus Rhamnosus GG (CGMCC 1.3724) is commercially available, for example, from Valio Oy. Bifidobacterium lactus BB-12 (CNCM I-3446) is commercially available, for example, from Christian Hansen.

The term probiotic as used herein refers to live probiotic bacteria, non-replicating probiotic bacteria, dead probiotic bacteria, non-viable probiotic bacteria, fragments of probiotic bacteria such as DNA, metabolites of probiotic bacteria, cytoplasmic compounds of probiotic bacteria, cell wall materials of probiotic bacteria, culture supernatants of probiotic bacteria, and/or combinations of any of the foregoing. The probiotic may for example be live probiotic bacteria, non-replicating probiotic bacteria, dead probiotic bacteria, non-viable probiotic bacteria, or any combination thereof. In an embodiment of the invention the probiotic is live probiotic bacteria.

Additional vitamins and minerals may also be administered in combination with scyllo-inositol. For example, the composition may contain one or more of the following micronutrients, calcium, magnesium, phosphorus, iron, zinc, copper, iodine, selenium, vitamin A or retinol activity equivalent (RAE) for example in the form of beta carotene or a mix of carotenoids, Vitamin C, Vitamin B1, niacin, folic acid, biotin, Vitamin E, vitamin B2, vitamin B6, vitamin B15, vitamin D, iron, zinc.

Vitamins and minerals may be administered in amounts in accordance with the recommendations of Government bodies such as the USRDA. For example: 100 to 2500 mg calcium, 35 to 350 mg magnesium, 70 to 3500 mg phosphorus, 2.7 to 45 mg iron, 1.1 to 40 mg zinc, 0.1 to 10 mg copper, 22 to 1,100 μg iodine, 6 to 400 μg selenium, 77 to 3000 μg of vitamin A or retinol activity equivalent (RAE) for example in the form of beta carotene or a mix of carotenoids, 8.5 to 850 mg Vitamin C, 0.14 to 14 mg Vitamin B1, 1.8 to 35 mg niacin, 60 to 1000 μg folic acid, 3 to 300 μg biotin, 1.9 to 109 μg Vitamin E.

In an embodiment of the invention, the scyllo-inositol is administered in combination with an ingredient selected from the group consisting of vitamin B2, vitamin B6, vitamin B12, vitamin D, magnesium, iron, zinc, arginine, glycine, serine or combinations thereof.

Vitamin B2 may for example be administered in an amount from 0.14 to 14 mg per day (daily dose). Vitamin B6 may for example be administered in an amount from 0.19 to 19 mg per day (daily dose). Vitamin B12 may for example be administered in an amount from 0.26 to 26 μg per day (daily dose). Vitamin D may for example be administered in an amount from 1.5 to 100 μg per day (daily dose). Magnesium may for example be administered in the form of MgCl₂ an amount of from 1 to 5 g per day (daily dose). Zinc may for example be administered in an amount of from 1.1 to 40 mg per day (daily dose). Arginine may for example be administered in an amount of from 2 to 30 g per day (daily dose). Glycine may for example be administered in an amount of from 5 to 20 g per day (daily dose). Serine may for example be administered in an amount of from 50 to 400 mg per kg per day (daily dose).

In a specific embodiment of the invention, the scyllo-inositol is administered in combination with vitamin B2, vitamin B6, Vitamin B12 and vitamin D, wherein said ingredients are administered in amounts equating to 1.8 mg of vitamin B2, 2.6 mg of vitamin B6, 5.2 μg of vitamin B12 and 10 μg of vitamin D per day.

In yet another embodiment the scyllo-inositol may be administered in combination with myo-inositol and/or other inositol isomers, for example selected from the group consisting of neo-inositol, epi-inositol, muco-inositol, allo-inositol and D-chiro-inositol.

Myo-inositol is known to be essential for cell functioning as it is important for cell growth, and to have insulin-mimetic effects with blood glucose lowering properties in an animal model of insulin resistance, and in women with polycystic ovary syndrome or with metabolic syndrome. D chiro-inositol is known to have blood glucose lowering properties via its insulin-mimetic effects.

In one embodiment of the present invention, the scyllo-inositol is administered in combination with D-chiro inositol.

A further embodiment of the present invention relates to scyllo-inositol for use in the treatment or prevention of disorders linked to an impaired function of insulin secreting β-cells and/or a condition or symptom associated with such disorders in a subject, wherein scyllo-inositol is to be administered in a composition that does not comprise other inositol isomers. For example, an embodiment of the present invention relates to scyllo-inositol for use in the treatment or prevention of disorders linked to an impaired function of insulin secreting β-cells and/or a condition or symptom associated with such disorders in a subject, wherein scyllo-inositol is to be administered in a composition that does not comprise myo-inositol. This can be beneficial in case the subjects do not tolerate other inositol isomers equally well.

In a further embodiment of the present invention, the scyllo-inositol is administered in the form of a composition. Said composition may comprise any other ingredient for example one or more ingredients set out herein e.g. probiotics vitamins and minerals. The composition may also comprise other ingredients commonly used in the form of composition in which it is employed e.g., a powdered nutritional supplement, a food product, or a dairy product. Non limiting examples of such ingredients include: other nutrients, for instance, selected from the group of lipids (optionally in addition to DHA and ARA), carbohydrates, and protein, micronutrients (in addition to those set out above), or pharmaceutically active agents; conventional food additives such as anti-oxidants, stabilizers, emulsifiers, acidulants, thickeners, buffers or agents for pH adjustment, chelating agents, colorants, excipients, flavor agents, osmotic agents, pharmaceutically acceptable carriers, preservatives, sugars, sweeteners, texturizers, emulsifiers, water and any combination thereof.

The composition may be any type of composition suitable for consumption for the subject to whom it is to be administered.

In an embodiment of the invention the composition is a product selected from the group consisting of a nutritional product, a food product, a functional food product, a healthy ageing product, a dairy product, a dairy alternative product, a nutritional supplement, a pharmaceutical formulation, a beverage product, a diet product, and a pet food product.

The term “nutritional product”, as used herein, means any product that can be used to provide nutrition to a subject. Typically, nutritional products contain a protein source, a carbohydrate source and a lipid source.

The term “food product”, as used herein, refers to any kind of product that may be safely consumed by a human or an animal. Said food product may be in solid, semi-solid or liquid form and may comprise one or more nutrients, foods or nutritional supplements. For instance, the food product may additionally comprise the following nutrients and micronutrients: a source of proteins, a source of lipids, a source of carbohydrates, vitamins and minerals. The composition may also contain anti-oxidants, stabilizers (when provided in solid form) or emulsifiers (when provided in liquid form).

The term “functional food product”, as used herein, refers to a food product providing an additional health-promoting or disease-preventing function to the individual.

The term “healthy ageing product”, as used herein, refers to a product providing an additional health-promoting or disease-preventing function related to healthy ageing to the individual.

The term “dairy products”, as used herein, refers to food products produced from milk or fractions of milk from animals such as cows, goats, sheep, yaks, horses, camels, and other mammals. Examples of dairy products are lowfat milk (e.g. 0.1%, 0.5% or 1.5% fat), fat-free milk, milk powder, whole milk, whole milk products, butter, buttermilk, buttermilk products, skim milk, skim milk products, high milk-fat products, condensed milk, crème fraiche, cheese, ice cream and confectionery products, probiotic drinks or probiotic yoghurt type drinks.

The term “dairy alternative product” refers to products similar to dairy products but produced without milk.

The term “milk” is defined by Codex Alimentarius as the normal mammary secretion of milking animals obtained from one or more milkings without either addition to it or extraction from it, intended for consumption as liquid milk or for further processing.

The term “pharmaceutical formulation” as used herein, refers to a composition comprising at least one pharmaceutically active agent, chemical substance or drug. The pharmaceutical formulation may be in solid or liquid form and can comprise at least one additional active agent, carrier, vehicle, excipient, or auxiliary agent identifiable by a person skilled in the art. The pharmaceutical formulation can be in the form of a tablet, capsule, granules, powder, liquid or syrup.

The term “beverage product” as used herein, refers to a nutritional product in liquid or semi-liquid form that may be safely consumed by an individual.

The term “diet product” as used herein, refers to a food product with a restricted and/or reduced caloric content.

The term “pet food product” as used herein refers to a nutritional product that is intended for consumption by pets. A pet, or companion animal, as referenced herein, is to be understood as an animal selected from dogs, cats, birds, fish, rodents such as mice, rats

The term “nutritional supplement” as used herein, refers to a nutritional product that provides nutrients to an individual that may otherwise not be consumed in sufficient quantities by said individual. For instance, a nutritional supplement may include vitamins, minerals, fiber, fatty acids, or amino acids. Nutritional supplements may for example be provided in the form of a pill, a tablet, a lozenge, a chewy capsule or tablet, a tablet or capsule, or a powder supplement that can for example be dissolved in water or sprinkled on food. Nutritional supplements typically provide selected nutrients while not representing a significant portion of the overall nutritional needs of a subject. Typically, they do not represent more than 0.1%, 1%, 5%, 10% or 20% of the daily energy need of a subject. A nutritional supplement may be used during pregnancy, e.g., as a maternal supplement.

As stated above, scyllo-inositol may be purchased or synthesized, for example. For some applications, it may be preferred, for example by consumers, if the scyllo-inositol is provided from natural sources. Scyllo-inositol may, for example, be present in plant or fruit concentrate e.g. coconut, grains such as buckwheat, and citrus. Scyllo-inositol is also present in in teleosts or other deep see animals. The scyllo-inositol may be extracted from these sources or may be employed in the form of these ingredients or an extract thereof. The skilled person would be able to calculate the amount of scyllo-inositol delivered by such extracts/ingredients on the basis of the concentration of scyllo-inositol in said extract/ingredient and the amount of the extract/ingredient employed. Hence, in one embodiment of the present invention, the scyllo-inositol is provided in the form of an extract from teleosts or other deep-sea animals. In another embodiment of the present invention, the scyllo-inositol is provided in the form of an extract from fruit, such as, for example, coconut, grains, such as buckwheat, citrus, and combinations thereof.

Those skilled in the art will understand that they can freely combine all features of the present invention disclosed herein. In particular, features described for the product of the present invention may be combined with the use of the present invention and vice versa. Further, features described for different embodiments of the present invention may be combined.

Although the invention has been described by way of example, it should be appreciated that variations and modifications may be made without departing from the scope of the invention as defined in the claims.

The term “and/or” used in the context of the “X and/or Y” should be interpreted as “X”, or “Y”, or “X and Y”.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 1 to 8, from 3 to 7, from 4 to 9, from 3.6 to 4.6, from 3.5 to 9.9, and so forth.

All references to singular characteristics or limitations of the present invention shall include the corresponding plural characteristic or limitation, and vice versa, unless otherwise specified or clearly implied to the contrary by the context in which the reference is made.

Unless defined otherwise, all technical and scientific terms used herein have the same meaning as commonly understood by one of ordinary skill in the art.

Furthermore, where known equivalents exist to specific features, such equivalents are incorporated as if specifically referred in this specification. Further advantages and features of the present invention are apparent from the figures and non-limiting examples.

### EXAMPLES

Experimental Conditions for Measuring In Vitro Insulin Secretion

INS-1 cellular model: The β pancreatic INS-1 cells were selected for their insulin secretion profile in response to glucose and to other physiological or pharmacological insulin secretagogues such as sulfonylureas and GLP-1. The cells were cultured in complete medium, RPMI 1640 11 mM glucose supplemented with 1 mM sodium pyruvate, 50 μM 2-mercaptoethanol, 2 mM glutamine, 10 mM HEPES, 100 IU/mL penicillin, 100 μg/mL streptomycin and 10% heat-inactivated foetal calf serum (FCS), as described by Asfari et al. (Endocrinology 130: 167-178, 1992). For the insulin secretion assay, INS-1 cells were plated and cultured in 96-well poly ornithine coated plates. The day before insulin secretion test, the medium were removed and replaced by RPMI 5 mM glucose supplemented with FCS 1%.

The day of the insulin secretion test, the cells were washed with Krebs-Ringer Bicarbonate HEPES buffer (KRBH; pH 7.4) 0.1% BSA and pre-incubated for 30 min at 37° C. in KRBH 0.1% BSA containing 2.8 mM glucose. The cells were then washed again with KRBH and incubated with the tested ingredients for 60 min in KRBH 0.1% BSA containing glucose (5 or 16 mM). The supernatants were collected and frozen before insulin determination.

The insulin concentration in the collected supernatants were measured by an ELISA kit according to the manufacturer recommendations and using a rat insulin antibody (Insulin rat high range ELISA Alpco Cat no 80-INSRTH-E10). Very briefly, rat monoclonal antibodies specific for insulin were immobilized to 96-well plates. Standards, samples and controls were added to the appropriate wells with a horseradish peroxidase enzyme-labeled monoclonal antibody (Conjugate). After incubation, the microplates were washed to remove unbound Conjugate and a TMB substrate solution was added to react with the bound Conjugate. Finally, after addition of a stop solution, the optical density was measured at 450 nm using a reference wavelength of 620 nm. The intensity of the yellow color is directly proportional to the amount of insulin within the samples. Human primary islets: Human islets from non-diabetic deceased male donors were purchased from Tebu-bio (Le-Perray-en-Yvelines, France). Islets were cultured in suspension at 37° C. in a humidified atmosphere (5% CO₂) in RPMI 1640 medium supplemented with 5.55 mM glucose, 10% (v/v) FCS, 10 mM HEPES pH 7.3, 1 mM sodium pyruvate, 50 μM β-mercaptoethanol, 1% v/v penicillin/streptomycin (all from ThermoFisher). All human islet procedures were approved by Commission cantonal d'étique de la recherche sur l'être humain (306/14).

Islets in suspension were equilibrated with Krebs-Ringer Bicarbonate HEPES buffer (KRBH; pH 7.4) for 1 hour at 37° C., followed by 1 h in 1 mM glucose. Clusters were then challenged for 1 h with 5 or 8 mM glucose in the presence or absence of 1 μM Scyllo-inositol or myo-inositol. Insulin in supernatants was determined with specific ELISA kits, according to the manufacturer's instructions (Ultrasensitive Insulin ELISA, ALPCO Diagnostics, Salem, USA). Cell pellets were lysed in 75% ethanol/0.55% HCl, followed by sonication. Insulin was measured in the cell lysates using specific ELISA kits (Ultrasensitive Insulin ELISA, ALPCO Diagnostics) and normalised to DNA content.

Results

The results of the study are shown in FIGS. 1-4.

The culture of INS-1 cells in the defined media appears to provide a useful model for studying insulin secretion in response to nutrient secretagogues such as glucose, GLP-1 and inositol isomers. Exposure of INS1-cells to 5 mM glucose, in the conditions described herewith, showed that insulin secretion (expressed as a percentage versus glucose) was higher than the glucose as control, FIG. 1. In addition, the most striking result is reported in FIG. 2 studying glucose stimulated insulin secretion (GSIS) with increase doses of scyllo-inositol from 20 to 2000 μM. By increasing the dose of scyllo-inositol from 20 to 2000 μM, GSIS increased from 120 to 142% reaching a plateau between 1000-2000 μM of scyllo-inositol.

Glucose stimulated insulin secretion tests was also tested on human primary islets in presence or absence of scyllo-inositol. Glucose at physiological concentrations (5 to 11.5 mM) stimulates insulin secretion from human β-cells. Exposure of human primary islets to glucose at 5 mM in presence of scyllo-inositol at a concentration of 1 μM showed a synergistic effect on insulin secretion potential over the control condition (5 mM glucose) (FIG. 3). Similar but lower effect was observed when exposing cells to 8 mM glucose and 1 μM of scyllo-inosito (FIG. 4). Indeed, the effect was more potent in condition of medium low glucose stimulation (5 mM) than higher concentrations (8 mM) but consistent. Scyllo-inositol at 1 μM consistently improves insulin secretion synergistically with glucose stimulation.

1. Method for use in the treatment or prevention of disorders linked to an impaired function of insulin secreting β-cells and/or a condition or symptom associated with such disorders in a subject comprising administering to the subject Scyllo-inositol.

2. Method in accordance with claim 1, wherein the disorder linked to an impaired β-cell function is selected from the group consisting of type 1 diabetes mellitus, type 2 diabetes mellitus, prediabetes, gestational diabetes mellitus, and polycystic ovary syndrome.

3. Method for use in accordance with claim 1, wherein the subject is a mammal.

4. Method in accordance with claim 1, wherein the subject is a female who is trying to get pregnant, is pregnant or who is lactating for her offspring.

5. Method in accordance with claim 1, wherein the scyllo-inositol is to be administered in an amount of 0.1 to 500 mg per day.

6. Method in accordance with claim 1, wherein the scyllo-inositol is to be administered to a subject in an amount of up to 0.001 g per kg body weight per day.

7. Method in accordance with claim 1, wherein the symptom associated with an impaired β-cell function is selected from the group consisting of un-usual hunger, increased thirst, un-usual bed-wetting, un-usual mood changes, irritability, fatigue, frequent urination, blurred eye sight, un-intended weight-loss, overweightness, obesity, and combinations thereof.

8. Method in accordance with claim 1, wherein the disorder associated with an impaired β-cell function is selected from the group consisting of nephropathy, heart disease, neuropathy, blood vessel disease, skin infections, complications during pregnancy, impaired vision due to damages in the blood vessels of the retina, foot complications, cardiovascular diseases, fatty liver diseases and combinations thereof.

9. Method in accordance with claim 1, wherein the scyllo-inositol is administered in combination with a probiotic selected from the group consisting of Lactobacillus rhamnosus GG (CGMCC 1.3724), Bifidobacterium lactus BB-12 (CNCM I-3446), and a combination thereof.

10. Method in accordance with claim 1, wherein the scyllo-inositol is administered in combination with an ingredient selected from the group consisting of vitamin B2, vitamin B6, vitamin B12, vitamin D, iron, zinc, arginine, glycine, serine and combinations thereof.

11. Method in accordance with claim 1, wherein the scyllo-inositol is administered in combination with myo-inositol and/or other inositol isomers.

12. Method in accordance with claim 1, wherein the scyllo-inositol is administered in combination with D-chiro inositol.

13. Method in accordance with claim 1, wherein the scyllo-inositol is administered in the form of a composition.

14. Scyllo-inositol for use in accordance with claim 13, wherein the composition is a product selected from the group consisting of a nutritional product, a food product, a functional food product, a healthy ageing product, a dairy product, a dairy alternative product, a nutritional supplement, a pharmaceutical formulation, a beverage product, a diet product, and a pet food product.

15. Method in accordance with claim 1, wherein the scyllo-inositol is provided in the form of an extract from teleosts and other deep-sea animals.
